(19) United States
(12) Patent Application Publication
(10) Pub. No.: US 2025/0302333 A1
(43) Pub. Date: Oct. 2, 2025

(54) ELECTRONIC MEASUREMENT SYSTEM FOR HUMAN FOOT LENGTH AND WIDTH AND HUMAN HEALTH MONITORING DEVICE

(71) Applicant: Airtouch Intelligent Technology (Shanghai) Co., Ltd., Shanghai (CN)

(72) Inventors: Shuiyang LIN, Shanghai (CN); Dekuan HE, Shanghai (CN); Jianhai YU, Shanghai (CN)

(73) Assignee: Airtouch Intelligent Technology (Shanghai) Co., Ltd., Shanghai (CN)

(21) Appl. No.: 18/900,753

(22) Filed: Sep. 29, 2024

(30) Foreign Application Priority Data
Mar. 29, 2024 (CN) 202410377781X

(51) Int. Cl. A61B 5/107; A61B 5/00

(52) U.S. Cl. CPC A61B 5/1074; A61B 5/1072; A61B 5/7475; A61B 2503/06

(57) ABSTRACT

An electronic measurement system for human foot length and width and a human health monitoring device. The system monitors the capacitance change values generated by the multiple touch points in contact with the subject's foot sole, and measures the subject's foot length and width based on the capacitance change values and the distribution positions of the touched points. The subject's foot length and width can be measured through the capacitance change region and values caused by the contact of the human foot with the multiple touch points. The measured results no longer have large measurement errors due to personal measurement method habits or inconsistent foot placement during children's measurements, etc., thereby significantly improving measurement accuracy and reducing costs.

### FIELD OF TECHNOLOGY

The present disclosure relates to the field of human body measurement technology, particularly an electronic measurement system for human foot length and width and a human health monitoring device.

### BACKGROUND

During children's growth period, the healthy development of their feet is critically related to their overall health development. Just like height and weight, foot data is an indispensable part of children's overall growth health. Regularly measuring and recording changes in children's foot length and width helps doctors monitor whether their growth and development are normal, detect potential problems such as skeletal dysplasia or obesity, thereby enabling timely and appropriate interventions. It also assists parents in selecting the appropriate shoe size for their children, preventing issues such as poor foot development or susceptibility to sport injuries due to inappropriate-size shoes.

Currently, there is no effective electronic measuring device for foot length and width on the market. Most measurements of children's foot length rely on physical measuring methods such as straight ruler or leather ruler measures. Due to differences in individual measurement habits and inconsistent children's foot placement during measurements, these physical measuring methods often result in significant errors, affecting the process of regular measurements.

### SUMMARY

The present disclosure provides an electronic measurement system for human foot length and width and a human health monitoring device to address measurement inaccuracy caused by differences in individual measurement habits and inconsistency from children's foot placement during measurements.

The electronic measurement system of human foot length and width includes the following elements: a touch sensing area distributed with multiple touch points, when the subject's foot is placed on the touch sensing area, certain touch points are contacted, inducing capacitance changes at internal circuits of these touch points; a touch chip module for real-time detection of touch points capacitance change values, is arranged to connect to each of the touch points and get capacitance change data from the contacted touch points that meets a minimum contact capacitance change requirement; a processing module connected to the touch chip module, receives capacitance data of all touch points from the touch chip module, and calculates the subject's foot information based on the contact capacitance changes, the subject's foot information including: foot length and foot width; a communication interface module, connected to the processing module, for reporting out the measurement data of the subject's foot.

In one embodiment of the present disclosure, the touch chip module includes one or more touch chips; each touch chip is connected to one or more touch points for real-time detection of the capacitance change value of the connected touch points and sending detected data of the contacted touch points that meet the requirements.

In one embodiment of the present disclosure, each touch chip includes a capacitance change detection module for real-time detection of capacitance data of each connected touch point, and detecting the capacitance change value of each touch point connected; a threshold judgment module for judging if the capacitance change value of a touch point exceeds the set threshold value thereby meeting the requirements; a data transmission module for sending to the processing module the detected data of each touch point that meet the requirements; the detected data includes: the capacitance change value and touch point distribution positions.

In one embodiment of the present disclosure, the processing module includes a foot print outline simulation module, that based on internal algorithms, simulates the foot print outline of the subject's foot sole by using the received capacitance change value of each contacted touch point and the distribution position of touch points; a foot data calculation module, connected to the foot print outline simulation module, for calculating the length and the width of the subject's foot based on the simulated foot outline by using human foot characteristics analysis algorithms.

In one embodiment of the present disclosure, the touch points are arranged in an array in the touch sensing area.

In one embodiment of the present disclosure, the touch points are arranged in a one-dimensional array on the touch sensing area; when subject's foot is placed along the direction of touch point arrangement on the touch sensing area, the length of subject's foot is measured using capacitance change data of the touch points contacted by the foot; when subject's foot is placed perpendicularly to the direction of touch point arrangement on the touch sensing area, the width of the subject's foot is measured using capacitance change data of the touch points contacted by the foot;

In one embodiment of the present disclosure, the touch points are arranged in a two-dimensional array in the touch sensing area; when the subject's foot is placed at any angle on the touch sensing area, the length and width of the subject's foot are simultaneously measured using capacitance change data of the touch points contacted by the foot.

In one embodiment of the present disclosure, the touch point is used one of FPC copper, a touch switch, PCB copper, and metal coated on non-conductors.

For the above purposes and other related purposes, the present disclosure provides a human health monitoring device capable of measuring the length and width of the human foot, including the human foot length and width electronic measurement system.

In one embodiment of the present disclosure, the human health monitoring device further includes a main control module connected to the human foot length and width electronic measurement system, which analyzes the foot health based on the foot measurement data of the subject's foot transmitted by the communication interface module.

As described above, the present disclosure provides an electronic measurement system for a human foot length and width and a human health monitoring device, with the following beneficial effects: the system monitors the capacitance change values generated by multiple touch points contacted with the subject's foot, and measures the length and width of subject's foot based on the capacitance change values of the contacted touch points and the distribution of touch points. The disclosure achieves the measurement of foot length and width by detecting the capacitance change region and magnitude caused by multiple touch points under the subject's foot sole, the measurement results are no longer affected by differences in individual measurement habits or inconsistent foot placement positions in children's foot measurements, thereby significantly improving foot measurement accuracy and reducing costs.

### BRIEF DESCRIPTION OF THE DRAWINGS

FIG. 1 shows a schematic block diagram of the human foot length and width electronic measurement system according to an embodiment of the present disclosure.

FIG. 2A shows a schematic diagram of foot length measurement in a one-dimensional array arrangement of touch points according to an embodiment of the present disclosure.

FIG. 2B shows a schematic diagram of foot width measurement in a one-dimensional array arrangement of touch points at different length locations according to an embodiment of the present disclosure.

FIG. 3 shows a schematic diagram of foot length and width measurement in a two-dimensional array arrangement of touch points according to an embodiment of the present disclosure.

FIG. 4 shows a schematic block diagram of a human foot length and width electronic measurement system according to an embodiment of the present disclosure.

FIG. 5 shows a schematic block diagram of a human health monitoring device capable of measuring human foot length and width according to an embodiment of the present disclosure.

FIG. 6 shows a schematic diagram of foot length and width measurement in a one-dimensional array arrangement of touch points on a weight (or fat) scale according to an embodiment of the present disclosure.

FIG. 7 shows a schematic diagram of foot length and width measurement in a two-dimensional array arrangement of touch points on a weight (or fat) scale according to an embodiment of the present disclosure.

### DETAILED DESCRIPTION

The embodiments of the present disclosure will be described below. Those skilled in the art can easily understand other advantages and effects of the present disclosure according to the contents disclosed by the specification. The present disclosure may also be implemented or applied through other different specific implementation modes. Various modifications or changes may be made to all details in the specification based on different points of view and applications without departing from the spirit of the present disclosure. It needs to be stated that the following embodiments and the features in the embodiments can be combined with one another under the situation of no conflict.

In the following description, referring to the accompanying drawings, which describe several embodiments of the present disclosure. It should be understood that other embodiments may be used and that changes in mechanical composition, structure, electrical, and operation may be made without departing from the scope of the present disclosure. The following detailed description should not be considered limiting, and the scope of the embodiments of the present disclosure is limited only by the claims of the patents. The terms used herein are for describing particular embodiments only, and are not intended to limit the present disclosure. Spatially related terms, such as “upper”, “lower”, “left”, “right”, “downward”, “below”, “bottom”, “above”, “top”, etc., can be used in the text for ease of explanation of the relationship between one element or feature and another element or feature shown in the figure.

Throughout the specification, when a component is “connected” with another component, this includes not only the “direct connection” but also the “indirect connection” in which other elements are placed therebetween. In addition, when a certain component “includes” a certain element, unless otherwise stated, other elements are not excluded, which means other elements may be included.

The terms first, second and third mentioned therein are used for the purpose of, but are not limited to, describing various parts, components, areas, layers and/or segments. These terms are used only to distinguish one part, component, area, layer or segment from other parts, components, areas, layers or segments. Accordingly, the first part, component, area, layer or segment described below may refer to a second part, component, area, layer or segment to the extent that it is not beyond the scope of the present disclosure.

In addition, as used herein, the singular forms “a”, “an” and “the” are intended to include the plural forms as well, unless the context clearly indicates otherwise. It should be further understood that the terms “comprise”, “include” indicate that there are the described features, operations, elements, components, items, categories, and/or groups, but the existence, appearance, or addition of one or more other features, operations, elements, components, items, categories, and/or groups are not excluded. The terms “or” and “and/or” are used herein to be interpreted as inclusive or meaning any one or any combination. Thus, “A, B or C” or “A, B and/or C” means “any of the following: A; B; C; A and B; A and C; B and C; A, B and C.” An exception to this definition occurs only when a combination of elements, functions or operations are inherently mutually exclusive in some manner.

For the measurement of human foot length and width, most current methods rely on physical measuring methods such as straight ruler or tape ruler measures, making it difficult to achieve precise measurements of foot length and width. The primary drawbacks of using physical measuring tools like a straight ruler or a tape ruler for measuring foot length and width are: first, due to differences in individual measurement habits and inconsistent foot placement during measurements especially in the case of children, there can be a significant margin of errors in the measurement results, making it challenging to achieve accurate and effective measurements of human foot length and foot width, thus affecting the significance of regular measurements; second, the measurement data of foot length and foot width cannot determine whether they fall within the normal range, still requiring further consultation with a health practitioner or specialist; third, the measurement data of foot length and foot width cannot be recorded and saved in real-time, making review and access historical records inconvenient when consulting with health practitioners or specialists.

The present disclosure provides a human foot length and width electronic measurement system. It monitors the capacitance change value in the measurement electronic system generated from multiple touch points under the subject's foot sole, and measures the subject's foot length and width based on the capacitance change value of the contacted touch points and the distribution positions of the touch points. The disclosure achieves the measurement of foot length and width by detecting the capacitance change region and magnitude caused by multiple touch points under the human foot sole, the measurement results no longer have large measurement errors due to personal measurement method habits or inconsistent foot placement during children's measurements, etc., thereby significantly improving measurement accuracy and reducing costs.

The embodiments of the present disclosure are described in detail below with reference to the drawings, so that those skilled in the art can easily implement the present disclosure. The present disclosure can be embodied in a variety of different forms and is not limited to the embodiments described herein.

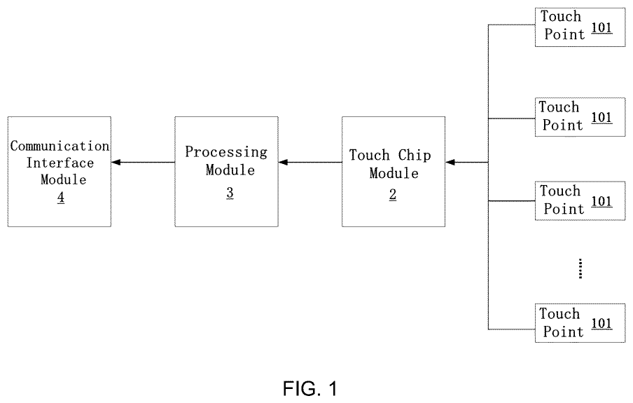

FIG. 1 shows a schematic block diagram of a human foot length and width electronic measurement system according to an embodiment of the present disclosure.

The measurement system includes:

A touch sensing area (not shown in the figure) with multiple touch points 101 distributed thereon; The touch sensing area is used to place human foot to be measured, the touch points 101 in the touch sensing area would generate capacitance change when they are contacted by human foot. When the subject's foot is placed in the touch sensing area, the contact between each touch point 101 and the relative touching foot location generates a capacitance change. This capacitance change can be converted into an electrical signal and accurately captured, which can be used for subsequent measurement and analysis. During the measurement process, when the subject's foot soles are placed on the touch-sensing area, due to the different shapes and sizes of different human feet, different numbers and positions of touch points 101 are contacted by human feet. Each contacted touch point will result in a different capacitance change based on the contact area and degree of contact proximity gaps. The size and shape of each touch point 101 can be set according to requirements, with shape such as square, rectangular, circular, and etc.

A touch chip module 2, connected with each touch point 101, is used to detect the capacitance change value of each touch point 101 in real-time and send the detected data of each contacted touch point that meets the contact requirements;

A processing module 3, connected with the touch chip module 2, for calculating the subject's foot data based on the detected data of the contacted touch points received from the touch chip module 2; and the subject's foot data includes: foot length and foot width; The processing module can be a microcontroller, CPU, DSP, ASIC, SoC and etc.

A communication interface module 4, connected with the processing module 3, for reporting the subject's foot measurement data Preferably, a serial port can be used to report the processed measurement data, and other protocol interfaces such as I2C/SPI/USB, or wireless methods such as BT/BLE/WIFI can be used to report the measurement information.

In an embodiment, the touch chip module 2 includes: one or more touch chips; each touch chip is connected with one or more touch points for real-time detection of the capacitance change value of the connected touch points and sending detected data of the contacted touch points that meet the requirements.

The detail cases are as following:
1. Adopt one touch chip with multiple touch channels: one touch chip connects with all touch points and detects the capacitance change values of all touch points in real time through multiple touch channels respectively.
2. Adopt multiple touch chips with a single touch channel: each of the touch chips is connected with a touch point; Then each touch chip detects the capacitance change value of the connected touch point in real-time through a single touch channel.
3. Adopt multiple touch chips with multiple touch channels or a single touch channel: one or more of the touch chips with multiple touch channels are connected with multiple touch points. The touch chip with multiple touch channels detects the capacitance change values of the connected touch points in real-time through multiple touch channels respectively. And the touch chip with a single touch channel detects the capacitance change value of the connected touch point in real-time through a single channel.

And, the touch chip can be optionally used according to the application situation, such as a self-capacitive touch chip, a mutual-capacitive touch chip, and other similar sensor chips.

In an embodiment, each touch chip includes:

A capacitance change detection module, is used to real-timely collect capacitance data of each connected touch point 101 and detect the capacitance change value of each connected touch point 101;

A threshold judgment module, is used to judge the touch point 101 with the capacitance change value exceeding the set threshold as a touch point that meets the requirements. Specifically, a threshold for capacitance change is typically set in order to avoid false touched judgment or noise interference. Only when the capacitance change value of the touch point exceeds the set threshold, the threshold judgment module would judge the touch point to be a contacted touch point that meets the requirements. In this way, noise or weak touch signals which below the threshold can be effectively filtered out to improve the accuracy of touch recognition.

A data transmission module, is used to send the detected data of each contacted touch point that meets the requirements to the processing module 3; the detected data includes: the capacitance change value and touch point distribution locations.

Preferably, as the present system is generally applied to a relevant health monitoring device, the health monitoring device is usually in standby mode when not in use, and only measures when the human foot is stepped on. Therefore, in order to save power, the touch chip will generate an interrupt signal to the processing module 3 as soon as it receives the detected data of a contacted touch point that meets the requirements, to wake up the processing module 3 to receive and process the detected data of each contacted touch point that meets the requirements.

In an embodiment, the processing module 3 includes:

A foot print outline simulation module, is used to simulate the foot outline of the subject's foot based on internal algorithms, according to the received capacitance change values and touch point distribution positions of each touched point; specifically, based on the received capacitance change values and the distribution of touch points, the relative position and direction of the foot on the sensing area can be detected, and the shape and size of the foot can be inferred, thereby simulating the outline of the subject's foot. The internal algorithm includes complex mathematical models and computer graphics techniques such as 3D modeling, surface fitting, image reconstruction and etc. To enhance the accuracy of the simulation, the algorithm may use machine learning techniques by training on a large amount of foot data to optimize model parameters. In this way, the algorithm can better understand and simulate foot sole outlines of different foot shapes and sizes.

a foot data calculation module, connected to the foot print outline simulation module, is used to calculate the length and width of the subject's foot based on the simulated foot print outline using human foot characteristics analysis algorithms. For example, the human foot characteristics analysis algorithm first identifies and locates key feature points of the foot, such as the heel, toe, and the widest part of the foot sole. The recognition of these feature points is the basis for calculating foot length and width. By analyzing the relative positions and distances of these feature points, the algorithm can determine the basic structure of the foot sole. Foot length generally refers to the straight-line distance from the heel to the toe, which is an important parameter for measuring foot length. The algorithm can calculate foot length by connecting the feature points of the heel and toe and computing the straight-line distance between these two points. Foot width, is the lateral distance of the widest part of the foot, which is usually located in the middle of the foot sole. The algorithm can determine foot width by identifying feature points at the widest part of the foot and calculating the distance between these points. The human foot characteristics analysis algorithm of the present disclosure can utilize various geometric and statistical methods such as least squares method, pattern recognition, machine learning, etc.

To better describe the distribution of touch points in the touch sensing area and the implementation structure of touch points, it is illustrated in conjunction with the following embodiments.

The touch points 101 of the present disclosure may be formed to be distributed on the touch sensing area in any arrangement. It can be arranged in arrays, other combinations of arrangements also can be used.

In one embodiment, each of the touch points 101 is arranged in an array on the touch sensing area. The distance interval between two adjacent touch points 101 can be set as desired.

In a specific embodiment, the touch points 101 are arranged in a one-dimensional array on the touch sensing area.

In the case where touch points 1˜N are arranged in a one-dimensional array; it is necessary to place the subject's foot twice in different directions to realize the measurement of foot length and width. Specifically, the two placements were: the subject's foot along the direction of the touch points and the subject's foot perpendicular to the direction of the touch points.

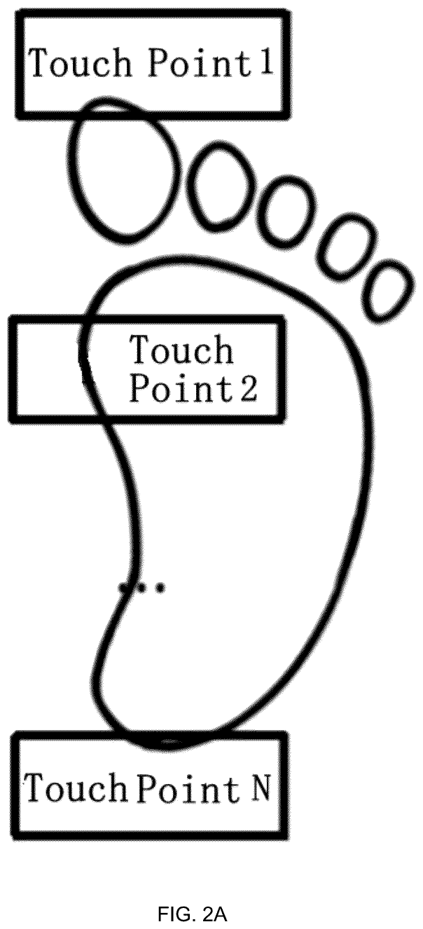

As shown in FIG. 2A, when the subject's foot is placed in the touch sensing area along the direction of the touch points arrangement, multiple touch points in the touch sensing area are contacted to generate capacitance change values, and the length of the human foot can be accurately measured based on the capacitance change value and distribution position of each contacted touch point that meets the requirements;

As shown in FIG. 2B, when the subject's foot is placed in the touch sensing area perpendicularly to the direction of the touch point arrangement, multiple touch points in the touch sensing area are contacted to generate capacitance change values, and according to the capacitance change value and the distribution position of each contacted touch point that meets the requirements, the width of the human foot can be accurately measured.

In a specific embodiment, the touch points 101 are arranged in a two-dimensional array on the touch sensing area; It is to be noted that the number of dimensions of the two-dimensional array and the interval of each touch point 101 are set according to the size of the touch points 101 and the standard human foot data, in order to realize that at least two touch points 101 can be touched in the foot-length direction or the foot-width direction no matter how the foot is placed.

In the case when touch points 1˜N are arranged in a two-dimensional array, only one placement of the human foot to be measured is needed to measure foot width and length. And subject's foot can be placed at any angle.

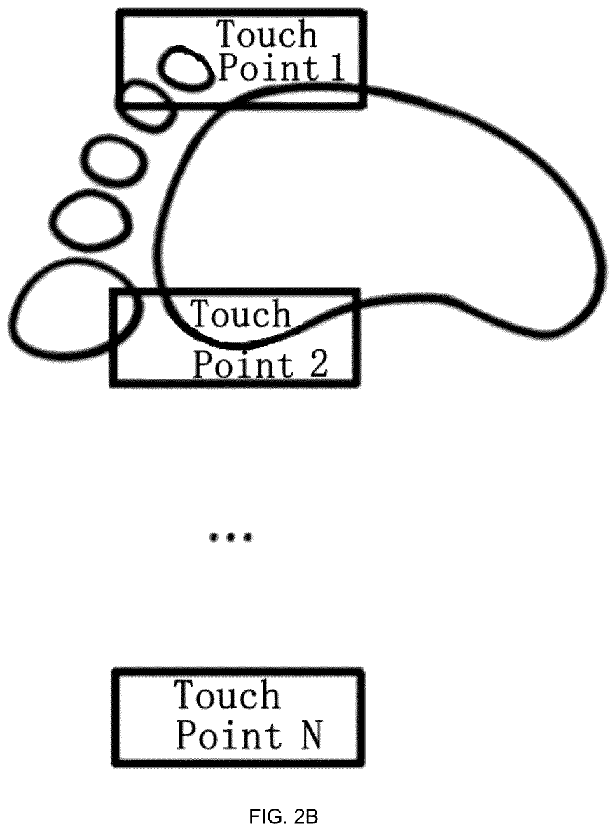

For example, as shown in FIG. 3, when the subject's foot is placed at an arbitrary angle in the touch sensing area, multiple touch points in the touch sensing area are contacted to generate capacitance change values, the foot length and foot width of the subject's foot can be measured simultaneously according to the capacitance change values and the distribution position of the contacted touch points that meet the requirements.

In an embodiment, when designing the touch point, the capacitance change caused by the contact or proximity of the human foot needs to be taken into account, and the sensitivity and stability of the touch points are ensured by reasonable material selection and design optimization.

One way is for touch points 101 to use a conductor material as one electrode of a capacitor, and with a non-conductive dielectric material coated on the conductor material as the dielectric of the capacitor. Due to conductive properties of human foot, it can be as the other electrode of the capacitor. When the human foot contacts or approaches the touch point 101, a parallel-plate capacitor is formed, thereby creating the capacitance change of the touch point 101. The capacitance change value is proportional to the area covered by the human foot at the relevant touch point 101 and the dielectric constant of the non-conducting dielectric material, and inversely proportional to the distance between the human foot and the relevant touch point 101.

The touch point 101 described in this way may adopt FPC copper, PCB copper, or metal coated on non-conductors.

Exemplarily, the FPC copper can be used as the touch point 101. FPC copper is a copper conductive layer formed on a flexible polyester or polyimide substrate through printed circuit technology. This material is thin, lightweight, soft and bendable with high reliability and excellent flexibility.

Specifically, the touch points 101 are made of FPC copper, embedded in the FPC, the FPC has two copper layers: the top side copper layer and the bottom side copper layer, and touch points 101 are located in the top side copper layer, in direct contact with the subject's foot, and to improve the sensitivity and accuracy of the touch points, the design of the touch points needs to take into account the size, shape and layout. The bottom side copper layer adjacent positions needs to be designed as keep out areas, which means that no conductive material can be placed in these areas to avoid forming a capacitor between touch point 101 and the bottom side copper layer's conductive material, which results in larger self-capacitance of the touch point 101, and when the human foot contacts the touch point 101, the capacitance change value is not large enough compared with the self-capacitance value, which affects the measuring sensitivity of the human foot. The FPC connection between the touch points and the touch chip is the critical path for signal transmission. To ensure stable signal transmission, special attention needs to be paid to clearance processing during the design of the connection wires, which includes ensuring that there is sufficient spacing between the connection wires to avoid cross interference among adjacent wires, as well as avoiding unnecessary contact between any wires and other parts of the FPC.

Similarly, the touch points 101 are made of PCB copper: PCB copper, as part of the PCB (printed circuit board), is a copper conductive layer formed on a rigid insulating substrate by chemical deposition or physical deposition methods. The touch points 101 are located on the top layer of the PCB, so the adjacent positions at the bottom layer need to be designed as keep out areas.

Similarly, the touch points 101 are made of metal coated on non-conductor, which means a metal layer formed on the surface of a non-conductor material (e.g., glass, plastic, ceramic, etc.) through electroplating or other methods. All touch points 101 are disposed on the surface of the non-conductor.

Another way is that touch switch also can be used as touch point 101: a touch switch is a switch that controls the on/off of a circuit according to capacitance change values when it is touched by a human. It usually consists of a touch sensor, a control circuit and an output interface.

To better describe the electronic measurement system for human foot length and width, specific examples are illustrated in conjunction with the embodiments.

### Embodiment: An Electronic Measurement System for Human Foot Length and Width

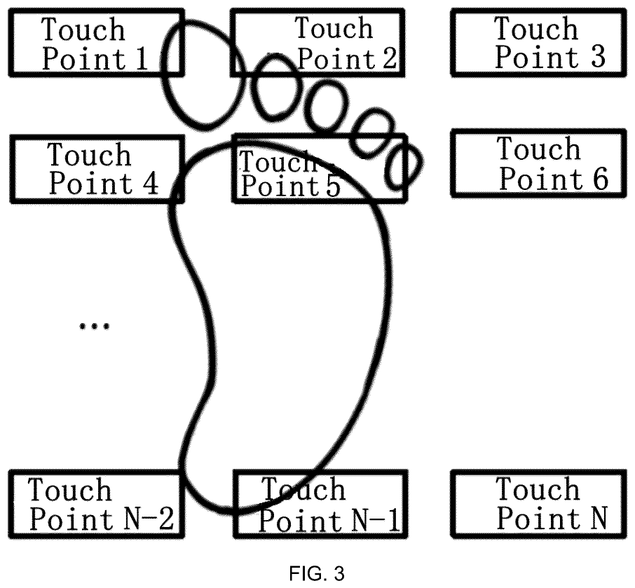

FIG. 4 is a schematic block diagram of the electronic measurement system for human foot length and width.

The system includes a serial port, a microprocessor, touch chips 1˜N, and touch points 1˜N;

The process of measuring the length and width of the human foot includes: when measuring, the human foot is placed on touch points 1 to N area. Depending on the size of the human foot, the part of touch points 1 to N that come into contact or approach the human foot will cause the capacitance changes due to the contact or approach of the human foot. Real-time monitoring of the capacitance change values of touch points 1 to N is carried out by touch chips 1 to N. When the capacitance change values of one or more touch points 1 to N caused by contact or approach of the human foot exceeds the set threshold, the corresponding touch chip detects that and generates an interrupt signal to the microprocessor, to wake it up to receive and process the capacitance change values of the touch points. By analyzing the distribution positions of the touch points and the corresponding capacitance change values through an internal algorithm, the microprocessor can simulate the foot print outline of the human foot, and combined with an internal human foot characteristics analysis algorithm, the length and width of the human foot can be measured.

The present disclosure provides a human health monitoring device that can measure human foot length and width. The human health monitoring device can be any device that monitors and records physiological parameters and health conditions of users in real time. Such as weight scales, body fat scales and foot health measuring instruments and other human health monitoring devices. The human health monitoring device includes: the electronic measurement system for human foot length and width as described in the above embodiment, which realizes the function of measuring human foot length and foot width.

The touch sensing area of the electronic measurement system for human foot length and foot width is the standing area of a human health monitoring device, such as the standing area of a weight scale or a body fat scale. When measuring human weight and body fat, it can also measure the human foot length and width.

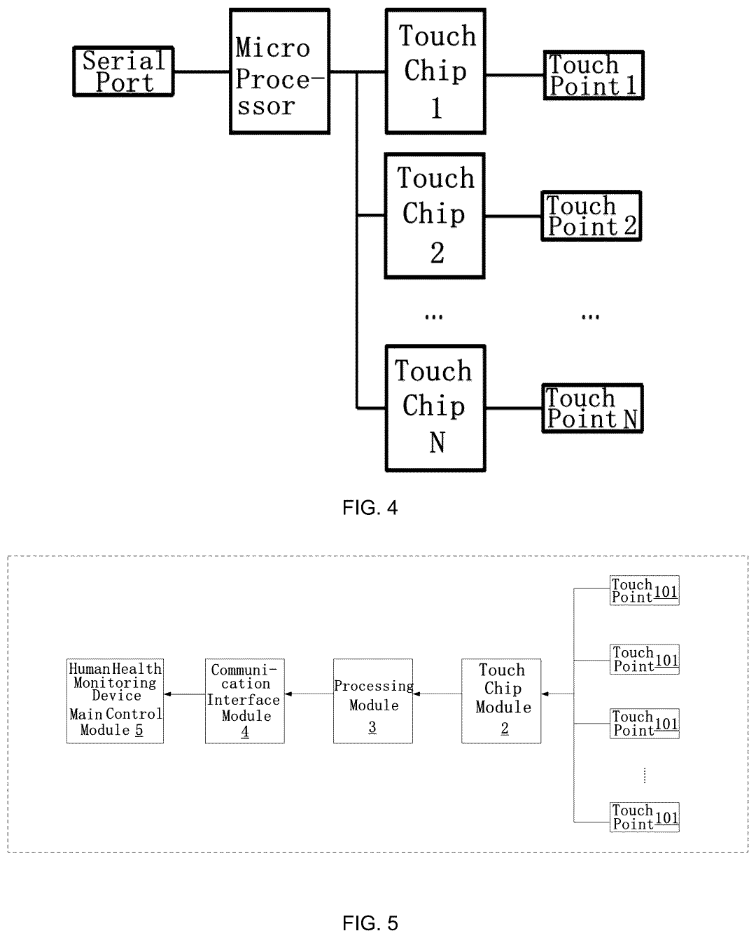

In an embodiment, as shown in FIG. 5, the human health monitoring device further includes: a main control module 5, connected to a communication interface module 4 of the electronic measurement system for human foot length and width;

When the subject's foot is placed in the touch sensing area, multiple touch points 101 are contacted to generate capacitance changes, and the touch chip module 2 detects the capacitance change value of each touch point in real-time and sends the detected data of the contacted touch points that meet the requirements; The processing module 3 measures the foot length and width of the subject's foot according to the detection data of the contacted touch points sent by the touch chip module 2, and then sends the measurement data to the main control module 5 of the human health monitoring device through the communication interface module 4, and the main control module 5 processes the measurement data, or controls the display of the measurement data.

The main control module 5 is also used to analyze the foot health based on the foot measurement data of the subject's foot transmitted by the communication interface module 4; Specifically, the main control module 5 is able to record, save, and analyze measurement data in real-time through an electronic health manager to assess whether the size of the human foot is healthy or whether the size of children's feet meets current age standards.

In a specific embodiment, the human health monitoring device is a weight scale or a body fat scale, and the touch sensing area is located in the human standing area of the scale.

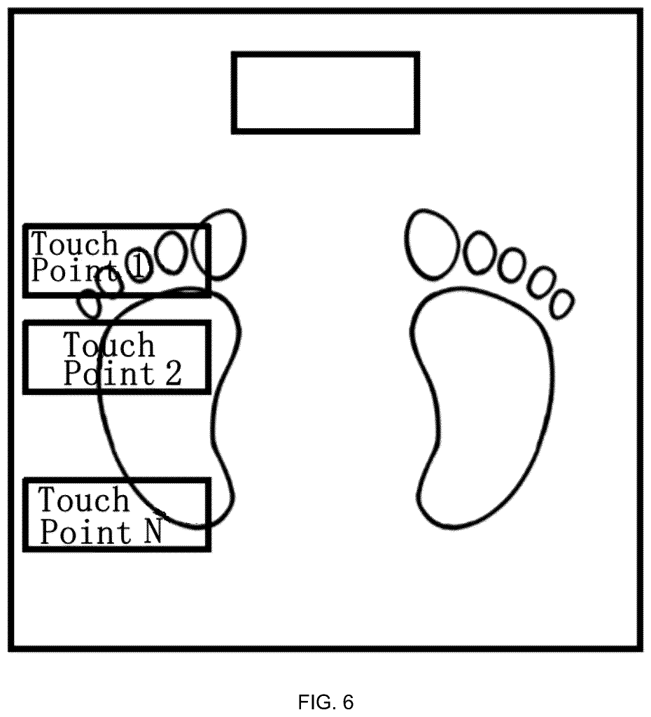

As shown in FIG. 6, the touch points are arranged in a one-dimensional arrangement distributed in the standing area of the body weight (fat) scale, when the subject's foot is placed along the direction of the arrangement of the touch points, the measurement system can accurately measure the length of the human foot through simple algorithms based on the capacitance change values of the touch points which contacted to the subject's foot and the position of the corresponding touch points; Similarly, when the human foot is placed perpendicularly to the direction of the one-dimensional touch point arrangement, the width of the human foot can also be accurately measured by a simple algorithm based on the capacitance change values of the touch points which contacted to the subject's foot and the position of the corresponding touch points.

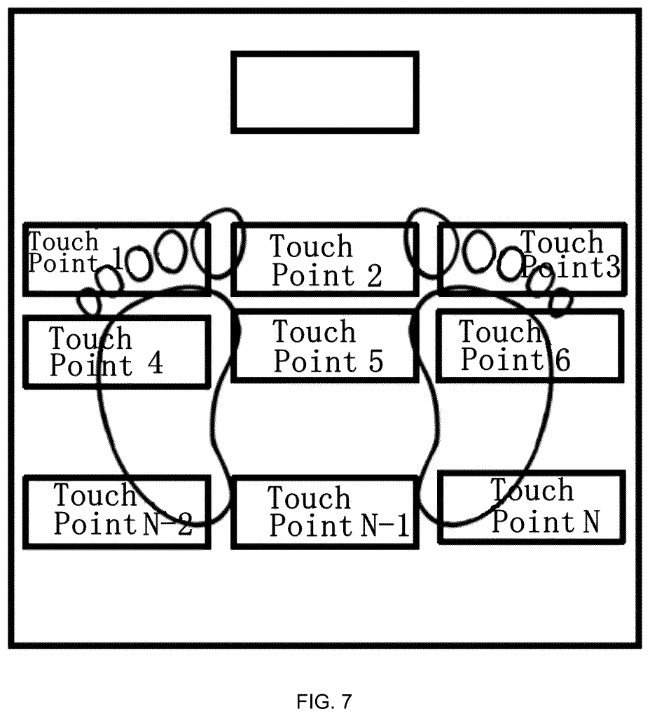

As shown in FIG. 7, the touch points are arranged in a two-dimensional arrangement distributed in the standing area of the body weight (fat) scale. When the subject's foot is placed anywhere within the range of touch points, the measurement system calculates the approximate foot print outline of the subject's foot based on the capacitance change values of the touch points which contacted to the subject's foot, and the position of the corresponding touch points. Combined with the internal human foot characteristics analysis algorithm, the subject's foot length and width can be measured.

In summary, the electronic measurement system for human foot length and foot width and the human health monitoring device of the present disclosure, monitor the capacitance change values generated by multiple touch points in contact with the subject's foot, and measure the foot length and width of the subject's foot based on the capacitance change values of the contacted touch points and the distribution positions of the touch points. The present disclosure achieves the measurement of foot length and width by detecting the capacitance change region and value caused by the contact of human foot with multiple touch points, and the measurement results no longer have large measurement errors due to personal measurement method habits, or inconsistent foot placement positions during children's feet measurements, etc., thereby significantly improving measurement accuracy and reducing costs. Therefore, the present disclosure effectively overcomes various shortcomings in the traditional technology and has high industrial utilization value.

The above-mentioned embodiments are merely illustrative of the principles and effects of the present disclosure, instead of limiting the present disclosure. Modifications or variations of the above-described embodiments may be made by those skilled in the art without departing from the spirit and scope of the disclosure. Therefore, all equivalent modifications or changes made by those skilled in the art without departing from the spirit and technical concept disclosed by the present disclosure shall be still covered by the claims of the present disclosure.

1. An electronic measurement system for human foot length and width, comprising:
a touch sensing area, distributed with multiple touch points, used to place a subject's foot, wherein each of the multiple touch points is one plate of a parallel-plate capacitor, and each of the multiple touch points on the touch sensing area generates capacitance changes based on a contact area and a distance between the multiple touch points and a subject's foot when the multiple touch points are contacted by the subject's foot, wherein each of the touch points is a touch switch, which controls on/off of a circuit according to capacitance change values when the touch switch is touched by a human;
a touch chip module, connected to each of the touch points, used to detect capacitance change values of the touch points in real-time, and output a detection data of the touched points that meet a threshold requirement, wherein the touch chip module comprises one or more touch chips;
a processing module, connected to the touch chip module, used to calculate a foot measurement data based on the detection data of the contacted touch points sent by the touch chip module, wherein, the foot measurement data includes: foot length and foot width; and
a communication interface module, connected to the processing module, used to report the foot measurement data of the subject's foot.

2. The electronic measurement system for human foot length and width according to claim 1, wherein each of the one or more touch chips is connected to one or more touch points with single or multiple channels for real-time detecting the capacitance change values of the contacted touch points.

3. The electronic measurement system for human foot length and width according to claim 2, wherein each of the one or more touch chips comprises:
a capacitance change detection module, used to acquire capacitance data of each touch point and detect the capacitance change values of each touch point in real-time;
a threshold judgment module, used to judge touch points whether the capacitance change values exceed a set threshold value of contacted touch points;
a data transmission module, used to send to the processing module the detection data of each touch point that meets the threshold requirement; wherein the detection data comprises: the capacitance change values and distributed positions of the touch points.

4. The electronic measurement system for human foot length and foot width according to claim 3, wherein the processing module comprises:
a foot print outline simulation module, used to simulate a foot print outline of the subject's foot sole based on internal algorithms, according to the received capacitance change values and distributed positions of the contacted touch points;
a foot data calculation module, connected to the foot print outline simulation module, used to calculate the foot length and width of the subject's foot based on the simulated foot print outline by using human foot characteristics analysis algorithms.

5. The electronic measurement system for human foot length and width according to claim 1, wherein the multiple touch points are arranged in an array on the touch sensing area.

6. The electronic measurement system for human foot length and width according to claim 5, wherein the multiple touch points are arranged in a one-dimensional array on the touch sensing area; wherein the touch chips connected to the multiple touch points are configured to detect the capacitance changes of the subject's foot placed parallel to the one-dimensional array of touch points for measuring the foot length, detect the capacitance changes of the subject's foot placed perpendicular to the one-dimensional array of touch points for measuring the foot width.

7. The electronic measurement system for human foot length and width according to claim 5, wherein the touch points are arranged in a two-dimensional array on the touch sensing area; wherein the touch chips connected to the touch points are configured to detect the capacitance changes of the subject's foot placed at any angle for measuring the foot length and the foot width simultaneously.

8. (canceled)

9. A human health monitoring device capable of measuring human foot length and width, comprising the electronic measurement system for the human foot length and width according to claim 1.

10. The human health monitoring device capable of measuring human foot length and width according to claim 9, wherein the device further comprises: a main control module, connected to the electronic measurement system for the human foot length and width, used to analyze foot health condition based on the subject's foot measurement data transmitted by the communication interface module.

11. (canceled)

12. (canceled)

13. (canceled)
